(19) United States
(12) Patent Application Publication
(10) Pub. No.: US 2011/0165025 A1
(43) Pub. Date: Jul. 7, 2011

(54) STOPPED-FLOW CHIP

(75) Inventors: Rainer Gransee, Mainz (DE); Thomas Hansen-Hagge, Mainz (DE); Friedhelm Schoenfeld, Mainz (DE); Frithjof van Germar, Muenster (DE); Nico Scharpfenecker, Hofheim (DE); Franz-Josef Meyer-Almes, Otzberg (DE)

(73) Assignee: INSTITUT FUER MIKROTECHNIK MAINZ GMBH

(21) Appl. No.: 12/999,219

(22) PCT Filed: Jun. 12, 2009
(86) PCT No.: PCT/EP09/04235

§ 371 Date: Mar. 4, 2011

(30) Foreign Application Priority Data
Jun. 18, 2008 (DE) 10 2008 002 509.7

(51) Int. Cl. G01N 21/00; G01N 33/00

(52) U.S. Cl. 422/82.05; 422/68.1

(57) ABSTRACT

A chip for performing and measuring chemical reactions, interactions (bonds), and/or conformational changes, especially fast chemical reactions and processes. The chip includes a base plate, which is made of a polymer material that is transparent at least in the measuring zone, and having. fluid ducts parallel to the plane of the base plate and includes at least two reagent feeders leading into a mixer structure that has a number of inlets corresponding to the number of reagent feeders and an outlet leading into a mixing section, the outlet of the mixing section leads into a measuring section, the outlet of the measuring section is connected to a discharge section, the discharge section leads out of the base plate or into a reservoir that is arranged on the chip, and pressure conduits lead into the reagent feeders at a distance from the mixer structure.

### CROSS REFERENCE TO RELATED APPLICATIONS

This claims priority from German Patent Application 10 2008 002 509.7 DE, filed Jun. 18, 2008.

### BACKGROUND OF THE INVENTION

The invention concerns a chip for carrying out and measuring chemical reactions, interactions (bonds) and/or conformational changes, especially fast chemical reactions and processes, which comprises a base plate comprising a polymer material which is transparent at least in the measurement region, and has fluid passages extending therein parallel to the plane of the base plate.

Stopped-flow apparatuses are usually employed to investigate very fast chemical and biochemical reactions, protein foldings and bonding reactions in the millisecond range. In that case generally the speed of a reaction which takes place after mixing of the reactants, with a color change, is observed. Frequently reactions of two reactants reacting with each other are investigated, but there can also be more. Known stopped-flow apparatuses are generally of a very complicated and expensive structure, they require a complex control system and they are therefore costly and susceptible to faults. The structure of most stopped-flow apparatuses used hitherto is very similar and differs only in a few details. The components to be caused to react with each other are disposed in separate storage containers, preferably syringes, and can be transferred into smaller working syringes by way of a hydraulic system. A plurality of measurement operations can be performed in succession by the use of storage containers. The actual stopped-flow reaction is initiated by the working syringes being simultaneously driven and the reaction partners being rapidly pressed into a mixing chamber and from there into an observation or measurement cuvette. The color change, fluorescence change, absorption change or change in circular dichroism, which take place in the measurement cuvette, by virtue of the proceeding reaction between the reaction partners, is measured and the time variation recorded. The flow of the reaction solutions through the mixing chamber and the measurement cuvette is abruptly stopped (stopped-flow), by again providing downstream of the outlet of the measurement cuvette a syringe for receiving the reaction solutions, the fluid reception of which is stopped or limited by an abutment. The moment in time at which the flow of the reaction solutions is abruptly stopped generally defines the commencement of measurement.

Modifications in the individual stopped-flow apparatuses lie in the nature in the actuating system and pressure transmission, and in respect of the choice of the suitable mixing chamber and the structure of the device for stopping the flow of fluid (triggering).

Stopped-flow apparatuses are used nowadays in many different ways in research. Besides investigating simple enzyme substrate reactions and the kinetics thereof for discovering and accounting for reaction mechanisms, many other kinetics are also detected, such as for example protein foldings, conformational changes on enzymes or other proteins. The stopped-flow process can also be used for example for observing substrate transport in vesicles and discovering and determining resulting intermediate products.

In a stopped-flow experiment the solution which is in the measurement cuvette when the fluid flow is stopped has already reacted to a certain amount. That is why the initial solutions cannot be infinitely quickly mixed with each other and the mixed solution still has to be transported from the mixing chamber into the measurement cuvette. The period of time from the first contact of the starting solutions to the start of the measurement is therefore referred as the dead time. It is an important quality criterion for stopped-flow apparatuses. Chemical reactions which take place within the dead time cannot be quantified by the measurement system. To determine the dead time, it is necessary to use a chemical reaction which takes place in part after but in part also within the dead time. If the measurement signal is known at the moment of mixing, that is to say at the beginning of the dead time, it is then possible to ascertain the dead time from extrapolation of the time variation, for example by pseudo-first order color reactions which occur exponentially and which can be linearized by a semi-logarithmic representation. The dead time can then be read off by extrapolation of the measurement value at the moment of starting to the zero value of the color reaction. The methods of determining dead time in stopped-flow apparatuses are known per se to the man skilled in the art. The dead time is a fixed apparatus constant and should therefore always be the same and is generally checked by renewed measurement at given time intervals. It can however also be ascertained separately for each experiment. The shorter the dead time of a stopped-flow apparatus, the correspondingly faster are the reactions which can be measured therewith.

With the development of modern microproduction technologies it has become possible to produce components of very small dimensions in the micrometer range with a high level of precision. Microfluidics is distinguished in that important basic operations in process engineering are carried out utilizing apparatuses with microstructured reaction regions and fluid passages, so-called microstructure apparatuses. The dimensions of the fluid structures, which are considerably reduced in comparison with conventional apparatuses, result in large specific surfaces and small diffusion paths, which in turn results in improved substance and heat transfer conditions. Microfluidics is a part of microprocess technology from which the development of lab-on-a-chip systems (laboratory on a chip) also resulted. In that respect the term microfluidics stands both for components and also for processes with which liquids and gases can be moved in the length range of below 1 mm, checked and analyzed. The uses extend from microdosage systems for medical active substances to completely miniaturized analysis systems. Liquids and gases are guided in micropassages, metered by microvalves and measured in respect of their through-flow and other chemical and physical properties.

### BRIEF SUMMARY OF THE INVENTION

The object of the present invention was that of providing a microfluidic stopped-flow chip which is less expensive to produce in comparison with known stopped-flow apparatuses, which requires fewer movable mechanical elements and which thus has a lower level of sensitivity to trouble and causes lower material and reagent costs.

In particular, the invention includes a chip for carrying out and measuring chemical reactions, interactions (bonds) and/or conformational changes, especially fast chemical reactions and processes, which comprises a base plate including a polymer material which is transparent at least in the measurement region, which base plate has fluid passages extending therein parallel to the plane of the base plate, with at least the following functional portions:

at least two reagent feed conduits

pressure conduits,

a mixer structure

a mixing section

a measurement section

an outlet section

wherein
the at least two reagent feed conduits open into the mixer structure,
the mixer structure has a number of inlets corresponding to the number of the reagent feed conduits and an outlet,
the outlet of the mixer structure opens into the mixing section, the discharge of the mixing section opens into the measurement section,
the discharge of the measurement section is connected to the outlet section,
the outlet section is passed out of the base plate or into a reservoir arranged on the chip, and

the pressure conduits open into the reagent feed conduits at a spacing in front of the mixer structure.

### BRIEF DESCRIPTION OF THE SEVERAL VIEWS OF THE DRAWINGS

FIG. 1 shows a diagrammatic view of an embodiment of the chip according to the invention from above,

FIG. 2 shows a diagrammatic view of a further embodiment of the chip according to the invention from above, and

FIG. 3 shows a diagrammatic view of an apparatus according to the invention with a chip as shown in FIG. 2 and components arranged outside the chip for conducting stopped-flow analysis processes.

### DETAILED DESCRIPTION OF THE INVENTION

The object according to the invention is attained by a chip for carrying out and measuring chemical reactions, interactions (bonds) and/or conformational changes, especially fast chemical reactions and processes, which comprises a base plate comprising a polymer material which is transparent at least in the measurement region, which base plate has fluid passages extending therein parallel to the plane of the base plate as previously discussed.

In a preferred embodiment of the invention the pressure conduits are passed out of the base plate for connection to a pressure reservoir by way of at least one connecting opening and/or the at least two reagent feed conduits are passed out of the base plate by way of connecting openings. Alternatively individual or all reservoirs can also be disposed on the stopped-flow chip itself, which for example can avoid the risk of contamination with dangerous reagents or products.

The stopped-flow chip according to the invention which hereinafter is simply referred to as the chip comprises a base plate of a polymer material which is transparent at least in the measurement region, with fluid conduit passages extending therein. It represents the central functional element of a stopped-flow apparatus. The essential fluid conduit processes, mixing processes and reactions take place in it. The chip according to the invention however is provided as a part or a replacement part of an overall stopped-flow apparatus which also includes an operator device for receiving the chip. The operator device desirably has a space for receiving the chip, in which the chip is either fixedly mounted or, and this is particularly preferred, can be easily replaceably fitted thereinto and removed therefrom again. The chip according to the invention can be in the form of a disposable or one-trip component for a single measurement or a limited number of measurements. Chips according to the invention of different structures for stopped-flow analysis procedures can be used in one and the same operator device. Depending on the nature of the reaction to be measured the fluid passages and the functional portions of the chips can be of different configurations.

An operator device for use with the chip according to the invention has at least one detector or a plurality of detectors for detecting light coupled out of the chip. The operator device can also have connections for a fluid communication with the fluid passages of the chip, in particular the reagent feed conduits, and also electronic components for evaluation and/or forwarding the signals received from the detectors, as well as chemical supplies, a fluid actuator system for conveying the fluids and a pressure reservoir such as for example a gas pressure container or a compressor, as well as one or more fast-switching valves. If the apparatus is to be used for carrying out absorption or transmission measurement procedures and/or fluorescence measurement procedures, the operator device desirably has one or more light sources for coupling light into the chip and optionally one or more filters which allow only certain wavelength ranges of light to pass through. The detectors for detecting light signals are per se known components which are commercially available. For example CCD chips are suitable for that purpose.

“Fluids” in accordance with the invention include gases and liquids but preferably liquids, particularly preferably liquids of a viscosity similar to that of water. Highly viscous liquids are less suitable as they can only be pressed through the micropassages of the chip with greater difficulty and more slowly than low-viscosity liquids. It is particularly advantageous for the functioning of the chip according to the invention that the reagents and the pressure fluids are not miscible with each other, for example aqueous reagents on the one hand and organic, apolar pressure fluids on the other hand, or have different phase states, for example liquid reagents on the one hand and gaseous pressure fluids on the other hand.

The term “transparent” in connection with the polymer material of the base plate of the chip according to the invention means that the material is permeable at least in the measurement region (in the region of the measurement section) at least for the light wavelengths to be measured in a stopped-flow experiment. There is no need for the polymer material to be transparent or permeable for example in the entire visible spectrum of light or in the non-visible wavelength range which can also be used for measurement operations. What is essential is the wavelength ranges of the coupled-in light and the coupled-out light, at which the measurement procedures are carried out.

A particularly advantage of the chip according to the invention is that the base plate of polymer material can be produced very inexpensively and with a high level of precision by shaping processes such as for example injection molding, hot embossing or reaction casting in the form of a one-piece shaped part.

In an embodiment the chip according to the invention also has on the base plate reflection surfaces which are so arranged that they deflect light coupled into the chip from a light source arranged outside the chip into the measurement section and/or couple light which is emitted and/or scattered by the measurement section out of the chip and preferably deflect it in the direction of a light detector in an operator device.

In the production of the chip according to the invention, preferably production processes and materials are so selected that the reflection surfaces are obtained without further post-working

The chip according to the invention includes at least two reagent feed conduits which in a preferred embodiment begin at an edge of the base plate and there have inlet openings. In operation of the stopped-flow apparatus those inlet openings of the reagent feed conduits are connected by way of connections to the storage containers for the reagents for carrying out the reaction to be measured. Those filling reservoirs can be syringe pumps or similar, as are known from conventional stopped-flow apparatuses. In an alternative embodiment however the reagent feed conduits can also be filled by capillary forces and/or gravitational forces.

The reagent feed conduits open into the mixer structure which has a number of inlets corresponding to the number of reagent feed conduits, and an outlet. There are many different possible design configurations for the mixer structure. In an embodiment of a chip in which the reaction of two reactants is to be measured, that is to say which has two reagent feed conduits, the mixer structure is a T-piece or Y-piece having two inlets and an outlet. Here the reagents are pressed in simultaneously through the two inlets, the fluid flows meet, in that case they are already highly intensively pre-mixed and they are discharged through the outlet into the adjoining mixing section. For thorough mixing, which is as complete as possible, of the reagents in the mixer structure, it is essential that a Reynolds number which is as high as possible is achieved at the intersection of the T-structure or Y-structure when the reagents come together, that is to say turbulent thorough mixing is guaranteed. Purely diffusive mixing at low Reynolds numbers is not suitable for mixing very fast reaction processes. To achieve a high Reynolds number a cross-section which is as small as possible is selected at the intersection of the T-mixer or Y-mixer, that cross-section enlarging again in the direction of the mixing section and the measurement section, to laminar flow conditions. Besides T- and Y-mixers, it is also possible to use other mixer structures as long as they provide for turbulent and very fast thorough mixing.

The outlet of the mixer structure opens into a mixing section which is desirably so selected that it also promotes very fast and intensive mixing of the reaction solutions, wherein the geometry of the mixing section has a considerable influence on the intensity and speed of mixing of the reactants. In an embodiment of the chip according to the invention the mixing section is of such a configuration that its cross-sectional area, starting from the connection to the outlet of the mixer structure, enlarges in the direction of its discharge where it opens into the measurement section. In other words, the internal space of the mixing section is enlarged from the outlet of the mixer structure towards the measurement section in a conical configuration. Alternatively it is also possible in accordance with the invention to use other geometries of the mixing section, such as for example mixing sections with so-called herringbone structures as are known from WO-A-03/011443 which are incorporated herein by reference.

In an embodiment the cross-sectional area of the mixing section increases, starting from the connection to the outlet of the mixer structure, in the direction of its discharge, to 2-fold the cross-sectional area or 3-fold the cross-sectional area or 4-fold the cross-sectional area or 5-fold the cross-sectional area or 10-fold the cross-sectional area.

The fluid passages of the chip according to the invention can be of any cross-section but preferably they are of a circular, semicircular, rectangular or square cross-section.

The cross-sectional area of the fluid passages can vary according to the purpose of use and function of the respective fluid passage portion. In an embodiment the cross-sectional area of the fluid passages is at least portion-wise in the range of between 0.05 and 4 mm² or between 0.1 and 3 mm² or between 0.25 and 2 mm².

Desirably the chip according to the invention has portions of the fluid conduit passages of larger cross-sectional area and those with a cross-sectional area that is reduced in comparison therewith. Portions of smaller cross-sectional area form for example stop structures for the fluids flowing in the chip.

In an embodiment of the invention the inlets of the mixer structure have a cross-sectional area smaller than half the cross-sectional area of the portion of the reagent feed conduit, that respectively leads into the inlet of the mixer structure. In a further embodiment the cross-sectional area of the inlet of the mixer structure is less than a third of the cross-sectional area or less than a quarter of the cross-sectional area or less than a fifth of the cross-sectional area or less than a tenth of the cross-sectional area of the portion of the reagent feed conduit, that leads into the inlet of the mixer structure.

The mixer structure includes a so-called “stop structure” which provides that the liquid in the reagent feed conduit, insofar as it is not under an increased pressure, is stopped at or in the mixer structure prior to contact and does not further flow thereinto. The “stop structure” is formed in the case of wetting fluids or reagents substantially by a passage constriction with an adjoining passage enlargement within the mixer structure (T-piece). In that case desirably the two inlets at the T-piece or Y-piece, with passage portions of small cross-section, respectively form a passage constriction. The passage enlargement is where the two inlet passages meet and form the T-shaped intersection with the outlet passage. If the liquid is not under increased pressure, the liquid remains at the end of the passage constriction or at the beginning of the passage enlargement by virtue of the surface tension. In the case of non-wetting fluids or reagents the stop structure is formed at the transition from a wide to a narrow passage portion.

Structures other than the above-described “stop structure” are also conceivable, such as for example diaphragms with vent openings which tear when a pressure is applied, or passage portions with differing surface wettability upstream of the mixer structure.

What is important for frictionless operation of the stopped-flow chip according to the invention is the avoidance of the formation of air bubbles and cavitation which make exact measurement difficult. What is helpful in that respect is the avoidance of sharp corners, edges and dead spaces. Transitions between changes in the cross-section of the passages are therefore preferably steady and without steps. Changes in the direction of the passages are desirably of a curved or at least rounded configuration. If wetting reagents are used, the surface of the fluid passages should be completely wetting except for possible stop structures.

At a spacing from the mixer structure the pressure conduits provided on the chip according to the invention open into the reagent feed conduits. The pressure conduits are connected to a pressure reservoir, wherein an increased pressure is not permanently applied to the pressure conduits but an increased pressure can be applied when required. If a branching pressure conduit is connected to a pressure reservoir then a pressure pulse can be synchronously delivered to a plurality of pressure conduits opening into the reaction feed conduits, by switching the pressure to the one pressure conduit, if the pressure conduits are of the same configuration.

In operation of the stopped-flow apparatus firstly the reagents, the reaction of which is to be measured after mixing thereof, are passed into the reagent feed conduits at least to the transition to the mixer structure. The reagent feed conduits are thus filled with reagent liquid in the portion between the inlet of the mixer structure and at least the location of the reagent feed conduits, at which the pressure conduits open laterally thereinto. The further flow of the reagents into the mixer structure, from there into the mixing section and further into the measurement section is initiated and caused by an increased gas pressure being applied to the pressure conduits, the gas pressure pressing the liquids in the reaction feed conduits into the mixer structure. In that case the reagent feed conduits can be desirably closed from the location of the reagent feed, in order to ensure a form for the pressure pulse on the reagents to be conveyed, that is as well defined as possible.

The gas pressure delivered to the pressure conduits is exactly controlled and delimited in respect of time so that the liquid pressed through the mixer structure and the mixing section flows into the measurement section and stops there, which is caused by removing or reducing the increased pressure. For that purpose a quick-switching valve, for example a piezo valve, in the chip or externally thereto, is used.

One of the essential differences and advantages of the chip according to the invention over the state of the art lies in the application of a gas pressure for rapidly introducing the fluids into the mixing and measurement devices. Known stopped-flow apparatuses use for that purpose a mechanically driven plunger syringe or plunger pump, which is slower and suffers from more inertia in comparison with the present invention. Applying a gas pressure for introducing the fluids however can be very exactly and quickly controlled for example by way of valves. In addition, the reagent feed conduits between the communication location and the mixer, which conduits can be very exactly produced by microtechnology processes, make it possible to achieve highly accurate dosing of the reagents using simple means.

A further essential difference of the invention over known stopped-flow apparatuses is that the known apparatuses have at the outlet of the measurement section a plunger syringe for receiving the liquid flowing out of the measurement section and for stopping the fluid flow. In those apparatuses the plunger is pushed out by the outwardly flowing liquid and the flow of liquid is stopped by the plunger for example encountering an abutment and thereby preventing a further discharge flow of fluid. That mechanical component is no longer required in the chip according to the invention as the fluid flow can be very accurately controlled by the application and removal of the pressure to the pressure conduits in conjunction with the microstructured passage dimensions.

The present invention has the further advantage over the state of the art that the amount of fluid required until the fluid flow stops can be less. There is no need as in the state of the art for the fluid to have to flow out of the measurement section for example into a plunger syringe until the flow of the fluid can be stopped mechanically. With the present invention pressure control can be so precise that the measurement section is just filled with the reaction mixture and no or only little fluid issues on the outlet side of the measurement section when the fluid flow is stopped. That makes it possible to carry out kinetic measurements with a considerably lesser reagent involvement than hitherto.

In an embodiment of the chip according to the invention provided at the lateral mouth openings of the pressure conduits into the reagent feed conduits are transitional portions of the pressure conduits of reduced cross-section, with a cross-sectional area which is less than half the cross-sectional area of the pressure conduits. In alternative embodiments the cross-sectional areas of the transitional portions are less than a third of the cross-sectional area or less than a quarter of the cross-sectional area or less than a fifth of the cross-sectional area or less than a tenth of the cross-sectional area of the pressure conduits. That reduction in the cross-sectional area at the transitional portions of the pressure conduits into the reagent feed conduits substantially provides that the liquid which has flowed in through the reagent feed conduits does not pass into the pressure conduits, or not to a substantial extent. The effect of those reduced transitional portions is similar to that of the stop structures at the transition from the reagent feed conduits into the mixer structure.

The chip according to the invention is described herein for explanatory purposes as a structure which is provided once and in which a simple set of fluid passage structures with the specified functional portions is provided in a single configuration in a base plate of transparent polymer material. The invention however is not limited thereto. The present invention also includes those chips in which the fluid passage structures with the functional portions are provided as a multiplicity in a single base plate or as part of a chip with otherwise different functional regions. The invention also embraces those embodiments in which a plurality of structural units comprising reagent feed conduits, mixer structure, mixing section, measurement section and outlet section as well as pressure conduits are provided on a base plate in parallel or in succession. With a parallel arrangement of such structural units on a base plate of plurality of reactions and measurement operations can be performed at the same time. The invention also embraces those chips in which only individual ones of the described functional portions are provided a plurality of times, such as for example a plurality of mixer structures, mixing sections and/or measurement sections, arranged parallel or in succession. For example a plurality of successive mixing sections can be provided to improve the nature and intensity of the mixing action. A parallel arrangement of a plurality of measurement sections downstream of the mixing section can be used for parallel implementation of different measurement processes on one and the same reaction, such as for example parallel measurement of absorption and fluorescence or parallel measurement of absorption or excitation with light of differing wavelengths.

The polymer material from which the base plate of the chip according to the invention is made is preferably selected from transparent acrylate, polymethylacrylate, polymethylmethacrylate, polycarbonate, polystyrene, polyimide, cycloolefin copolymer (COC), cycloolefin polymer (COP), polyurethane, epoxy resin, halogenated acrylate, deuterated polysiloxane, PDMS, fluorinated polyimide, polyetherimide, perfluorocyclobutane, perfluorovinylether copolymer (Teflon AF), perfluorovinylether cyclopolymer (CYTOP), polytetrafluoroethylene (PTFE), fluorinated polyarylethersulfide (FRAESI), inorganic polymer glass, polymethylmethacrylate copolymer (P2ANS).

The term “polymer material” is also used in accordance with the present invention to denote glasses which are suitable for production of the chip according to the invention.

The present invention embraces not only the chip comprising a base plate of a transparent polymer material, but also an overall apparatus for carrying out and measuring chemical reactions, which includes both the chip according to the invention and also an operator device which has a space for receiving the chip, at least one light source for coupling light into the chip and a detector or a plurality of detectors for the detection of light which is coupled out of the chip.

In a further embodiment there is further provided on the apparatus according to the invention at least one pressure reservoir for providing a gas pressure and connections for connecting the pressure reservoir to the pressure conduits of the chip.

In a further embodiment of the apparatus according to the invention there are further provided reagent reservoirs for providing liquid or gaseous reagents and connections for connecting the reagent reservoirs to the reagent feed conduits of the chip.

Further advantages, features and possible uses will be apparent from the description hereinafter of some preferred embodiments and the accompanying Figures.

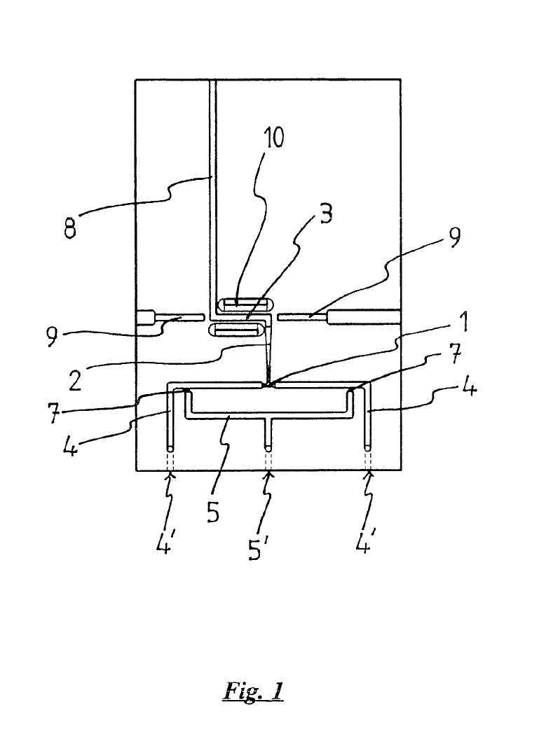

FIG. 1 shows a diagrammatic view of a chip according to the invention comprising a base plate of a transparent polymer material. In this example the base plate is made from polymethylmethacrylate (PMMA). The chip as shown in FIG. 1 includes two reagent feed conduits 4 having inlet openings 4′ at the edge of the base plate for the connection to reagent reservoirs. In this embodiment the two reagent feed conduits 4 are symmetrical and are of the same cross-section with a cross-sectional area of about 2.5 mm². There is also a pressure conduit 5 having an inlet opening 5′ at the edge of the base plate for connection to a pressure reservoir. The pressure conduit 5 is divided into two portions which open into a respective one of the two reagent feed conduits 4 at a spacing from the mixer structure 1. Provided at the lateral mouth openings of the pressure conduits 5 into the reagent feed conduits 4 are transitional portions 7 of reduced cross-section, which are intended to prevent fluid from flowing out of the reagent feed conduits 4 into the pressure conduits 5. The reagent feed conduits 4 open into a T-shaped mixer structure 1 which, corresponding to the number of reagent feed conduits 4, has two inlets and an outlet. The mixer structure 1 includes a so-called “stop structure”. At the inlets that includes firstly fluid passages of a cross-sectional area that is markedly smaller than the reagent feed conduits. The cross-sectional area of those fluid passages of the mixer structure 1 is approximately one fifth of the cross-sectional area of the fluid passages of the reagent feed conduits. The passage cross-section becomes wider again at the point of intersection of inlet passages with the outlet of the T-shaped mixer structure 1. That transition from the narrow passage cross-section to the passage enlargement represents the actual “stop structure” (see above).

The outlet of the mixer structure 1 opens into a mixing section 2, the internal geometry of which corresponds to an enlarging cone, wherein the cross-sectional area at the entry of the mixing section 2 corresponds to that at the outlet of the mixer structure 1 and the cross-sectional area at the discharge of the mixing section 2 corresponds to that at the entry or the feed line of the adjoining measurement section 3. The mixing section 2 opens into the measurement section 3 in which kinetic measurement of the reaction to be investigated takes place.

Arranged around the measurement section 3 in the base plate are openings, for example in the form of bores 9 or passages for the introduction of optical fibers, by way of which light can be coupled from outside the chip into the measurement section or out of the measurement section out of the chip. Arranged laterally of the measurement section 3 on the base plate are reflection surfaces 10 extending substantially parallel to the measurement section 3. The reflection surfaces are preferably arranged at an angle of 45° relative to the base plate and serve to deflect light which is emitted laterally from the measurement section such as for example fluorescence radiation or scatter light onto a detector or light receiver arranged beneath the base plate of the chip.

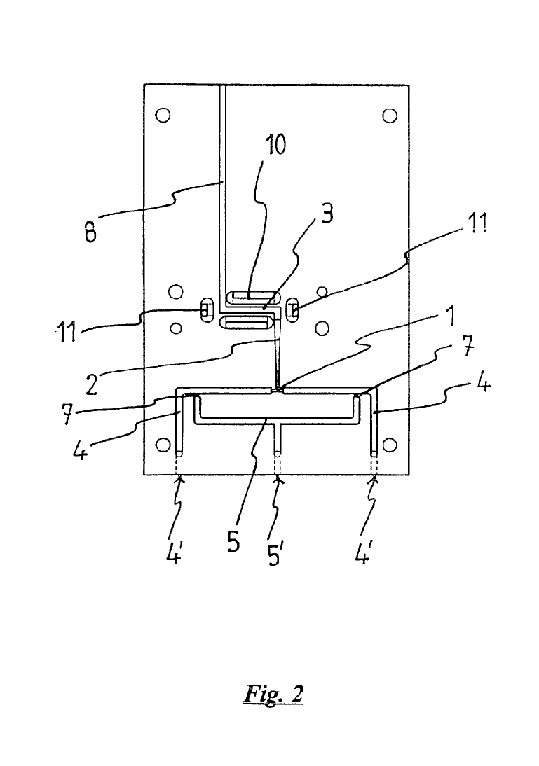

FIG. 2 shows an alternative embodiment of the chip according to the invention which only differs from the embodiment of FIG. 1 in that, instead of the bores 9 provided in the FIG. 1 chip for the introduction of optical fibers, here coupling-in and coupling-out mirror surfaces 11 are provided. The coupling-in and coupling-out mirror surfaces 11 are similar to the reflection surfaces 10 but at the entry and exit sides of the measurement section 3. Light which is incident on one of the coupling-in and coupling-out mirror surfaces 11 from a source for excitation light beneath the base plate is deflected into the measurement section 3. When the light issues from the opposite side of the measurement section 3 again it is then incident on the second one of the coupling-in and coupling-out mirror surfaces 11 and is again deflected substantially perpendicularly out of the base plate. In other respects the embodiments of the chip shown in FIGS. 1 and 2 are the same, and for that reason the same components are also denoted by the same references.

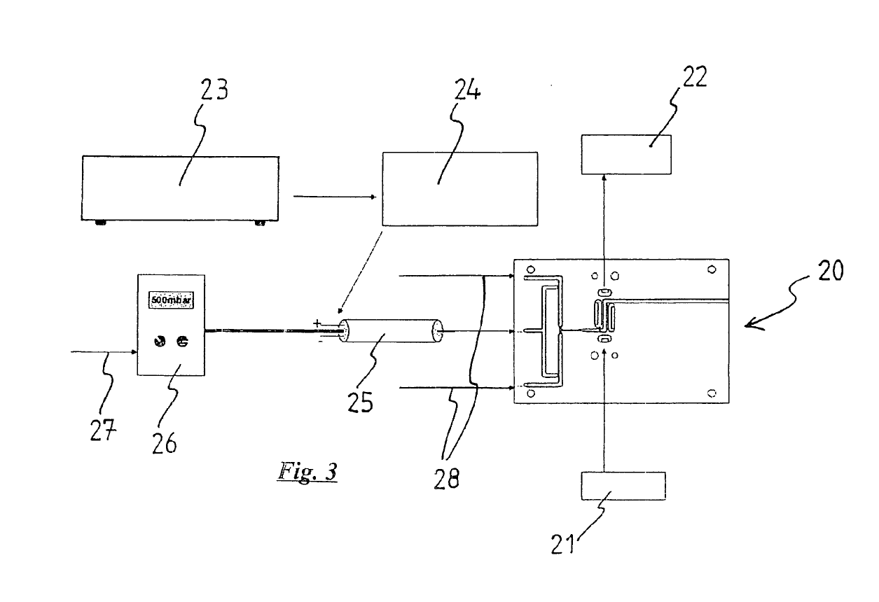

FIG. 3 shows a diagrammatic view of an apparatus according to the invention with a chip as shown in FIG. 2 and some components required for carrying out stopped-flow analyses in addition to the chip. In FIG. 3 those components are illustrated as being arranged outside the chip, but individual ones of the components in other embodiments can also be integrated on or in the chip.

The apparatus of FIG. 3 firstly includes a source 21 for excitation light, from which excitation light is incident on a coupling-in mirror surface on the chip and is deflected thereat into the measurement section on the chip. On the opposite side of the measurement section the light which has passed through the measurement section is incident on a coupling-out mirror surface which again deflects the light out of the base plate of the chip onto a receiver and a measuring device 22 for coupled-out light. The apparatus further includes a mains power unit 23 and a device having an electronic actuation arrangement 24 which actuates at least the high-speed valve 25. The apparatus further includes a pressure regulator 26 which feeds compressed air which is fed in by way of a compressed air feed 27 to the high-speed valve 25. The apparatus further includes reagent and sample feed means 28 by way of which reagents and/or samples are introduced into corresponding inlets of the reagent feed conduits on the chip.

### LIST OF REFERENCES

1 mixer structure
2 mixing section
3 measurement section
4 reagent feed conduits
4′ inlets of the reagent feed conduits
5 pressure conduits
5′ inlets of the pressure conduits
7 transitional portions
8 outlet section
9 optical fiber bores
10 reflection surfaces
11 coupling-in and coupling-out mirror surfaces
20 chip as shown in FIG. 2
21 source for excitation light
22 receiver and measuring device for coupled-out light
23 mains power supply
24 electronic actuation arrangement
25 valve
26 pressure reservoir
27 compressed air feed means
28 reagent and sample feed means

What is claimed is:

1-13. (canceled)

14. A chip for carrying out and measuring chemical reactions, interactions (bonds) and/or conformational changes, especially fast chemical reactions and processes, which comprises a base plate comprising a polymer material which is transparent at least in the measurement region, which base plate has fluid passages extending therein parallel to the plane of the base plate, with at least the following functional portions:
at least two reagent feed conduits (4),
pressure conduits (5),
a mixer structure (1),
a mixing section (2),
a measurement section (3),
an outlet section (8),
wherein
the at least two reagent feed conduits (4) open into the mixer structure (1),
the mixer structure (1) has a number of inlets corresponding to the number of the reagent feed conduits (4) and an outlet,
the outlet of the mixer structure (1) opens into the mixing section (2),
the discharge of the mixing section (2) opens into the measurement section (3),
the discharge of the measurement section (3) is connected to the outlet section (8),
the outlet section (8) is passed out of the base plate or into a reservoir arranged on the chip, and
the pressure conduits (5) open into the reagent feed conduits (4) at a spacing in front of the mixer structure (1).

15. A chip as set forth in claim 14 wherein the pressure conduits (5) are passed out of the base plate for connection to a pressure reservoir by way of at least one connecting opening and/or the at least two reagent feed conduits (4) are passed out of the base plate by way of connecting openings.

16. A chip as set forth in claim 14 wherein reflection surfaces (10) are provided on the base plate arranged that they deflect light coupled into the chip from a light source arranged outside the chip into the measurement section (3) and/or couple light emitted and/or scattered in the measurement section (3) out of the chip and preferably deflect it in the direction of a light detector provided in an operator device.

17. A chip as set forth in claim 15 wherein reflection surfaces (10) are provided on the base plate arranged that they deflect light coupled into the chip from a light source arranged outside the chip into the measurement section (3) and/or couple light emitted and/or scattered in the measurement section (3) out of the chip and preferably deflect it in the direction of a light detector provided in an operator device.

18. A chip as set forth in claim 14 wherein the fluid passages are of a circular or semicircular cross-section.

19. A chip as set forth in claim 14 wherein each of the inlets of the mixer structure (1) has a cross-sectional area which is smaller than half the cross-sectional area of a portion of the reagent feed conduit (4), that opens into the inlet of the mixer structure (1).

20. The chip of claim 19 where the inlets of the mixer structure has a cross section less than ¼ of the cross-sectional area of the cross-sectional area of a portion of the reagent feed conduit (4), that opens into the inlet of the mixer structure (1).

21. The chip of claim 19 where the inlets of the mixer structure has a cross section less than 1/10 of the cross-sectional area of the cross-sectional area of a portion of the reagent feed conduit (4), that opens into the inlet of the mixer structure (1).

23. A chip as set forth in claim 14 wherein the cross-sectional area of the mixing section (2) increases in size starting from the connection to the outlet of the mixer structure (1) in the direction towards its discharge where it opens into the measurement section (3), at least 2-fold times the cross-sectional area.

24. A chip as set forth in claim 14 wherein the cross-sectional area of the mixing section (2) increases in size starting from the connection to the outlet of the mixer structure (1) in the direction towards its discharge where it opens into the measurement section (3), at least 4-fold times the cross-sectional area.

25. A chip as set forth in claim 14 wherein the cross-sectional area of the mixing section (2) increases in size starting from the connection to the outlet of the mixer structure (1) in the direction towards its discharge where it opens into the measurement section (3), at least 10-fold times the cross-sectional area.

26. A chip as set forth in claim 14 wherein transitional portions of the pressure conduits (5) are provided at lateral mouth openings of the pressure conduits (5) into the reagent feed conduits (4) that are of reduced cross-section, with a cross-sectional area which is less than half cross-sectional area of the pressure conduits (5) upstream of said transitional portions, wherein transitional portions are less than ⅓ of the cross-sectional area of the pressure conduits (5) upstream of said transitional portions.

27. A chip as set forth in claim 14 wherein transitional portions of the pressure conduits (5) are provided at lateral mouth openings of the pressure conduits (5) into the reagent feed conduits (4) that are of reduced cross-section, with a cross-sectional area which is less than half cross-sectional area of the pressure conduits (5) upstream of said transitional portions, wherein transitional portions are less than ⅕ of the cross-sectional area of the pressure conduits (5) upstream of said transitional portions.

28. A chip as set forth in claim 14 wherein there are provided precisely two reagent feed conduits (4) and the mixer structure (1) is in the form of a T-piece mixer.

29. A chip for parallel performance and measurement of a plurality of chemical reactions, wherein the fluid passage structures with the functional portions of claim 14 are provided a plurality of times in a base plate of transparent polymer material.

30. A chip as set forth in claim 14 wherein the base plate is made from transparent acrylate, polymethylacrylate, polymethylmethacrylate, polycarbonate, polystyrene, polyimide, cycloolefin copolymer (COC), cycloolefin polymer (COP), polyurethane, epoxy resin, halogenated acrylate, deuterated polysiloxane, PDMS, fluorinated polyimide, polyetherimide, perfluorocyclobutane, perfluorovinylether copolymer (Teflon AF), perfluorovinylether cyclopolymer (CYTOP), polytetrafluoroethylene (PTFE), fluorinated polyarylethersulfide (FRAESI), inorganic polymer glass, polymethylmethacrylate copolymer (P2ANS).

31. Apparatus for performing and measuring chemical reactions, interactions (bonds) and/or conformational changes, in particular fast chemical reactions and processes, having a chip as set forth in claim 14 and an operator device that has a space for receiving the chip, at least one light source for coupling light into the chip and at least one detector for the detection of light which is coupled out of the chip (1).

32. Apparatus as set forth in claim 31 wherein at least one pressure reservoir is provided for providing a gas pressure and connections for connecting the pressure reservoir to the pressure conduits (5) of the chip.

33. Apparatus as set forth in claim 31 wherein reagent reservoirs are further provided for providing liquid or gaseous reagents and connections for connecting the reagent reservoir to the reagent feed conduits (4) of the chip.
